(19) United States
(12) Patent Application Publication
(10) Pub. No.: US 2023/0169312 A1
(43) Pub. Date: Jun. 1, 2023

(54) ARTIFICIAL INTELLIGENCE SYSTEM DECODING USER'S THOUGHTS AND METHOD FOR CONTROLLING THEREOF

(71) Applicant: 4N Inc., Daejeon (KR)

(72) Inventors: Seongkyun KIM, Seoul (KR); Jaehyung KWON, Daejeon (KR); Hoon-Hee KIM, Daejeon (KR)

(73) Assignee: 4N Inc., Daejeon (KR)

(21) Appl. No.: 18/047,073

(22) Filed: Oct. 17, 2022

(30) Foreign Application Priority Data
Nov. 30, 2021 (KR) 10-2021-0167808
Oct. 7, 2022 (KR) 10-2022-0128484

(51) Int. Cl. G06N 3/0455; G06N 3/044; G06N 3/08; G06F 40/279

(52) U.S. Cl. CPC G06N 3/0455; G06N 3/044; G06N 3/08; G06F 40/279; G06F 40/30

(57) ABSTRACT

Disclosed is an artificial intelligence system including a processor that preprocesses a measured biometric signal, extract at least one first biometric signal feature from the preprocessed biometric signal, determines at least one second biometric signal feature necessary to identify thoughts of the user among the at least one first biometric signal feature by using a first machine learning model learned to identify thoughts of the user, learns a second machine learning model while using the at least one second biometric signal as an input and using a word constituting the thoughts of the user as an output in the learned first machine learning model, and derives at least one word constituting the thoughts of the user by using the learned first machine learning model and the learned second machine learning model.

### CROSS-REFERENCE TO RELATED APPLICATIONS

A claim for priority under 35 U.S.C. § 119 is made to Korean Patent Application Nos. 10-2021-0167808 and 10-2022-0128484 filed on Nov. 30, 2021 and Oct. 7, 2022 in the Korean Intellectual Property Office, the entire contents of which are hereby incorporated by reference.

### BACKGROUND

Embodiments of the inventive concept described herein relate to an artificial intelligence system for decoding a user's thoughts, and more particularly, relate to an artificial intelligence system using an artificial intelligence model and a natural language processing scheme.

With the development of an artificial intelligence technology, many changes have occurred in human life. As an artificial intelligence system evolves through learning, what was previously considered impossible is gradually being realized.

Accordingly, many researchers are taking on challenges, which have not been solved before, by using the artificial intelligence technology. Understanding human thoughts through biometric signals of human is also one of these challenges.

### SUMMARY

Embodiments of the inventive concept provide an artificial intelligence system for decoding a user's thoughts that derives words constituting the user's thoughts by using an artificial intelligence model and a natural language processing scheme, converts the words into a machine command for controlling a user terminal, and executes the machine command on the user terminal.

According to an embodiment, an artificial intelligence system includes a processor that preprocesses a measured biometric signal, extract at least one first biometric signal feature from the preprocessed biometric signal, determines at least one second biometric signal feature necessary to identify thoughts of the user among the at least one first biometric signal feature by using a first machine learning model learned to identify thoughts of the user, learns a second machine learning model while using the at least one second biometric signal as an input and using a word constituting the thoughts of the user as an output in the learned first machine learning model, and derives at least one word constituting the thoughts of the user by using the learned first machine learning model and the learned second machine learning model.

In some embodiments of the inventive concept, the processor may convert the derived at least one word into a machine command for controlling a user terminal by using a natural language processing scheme (NLP).

In some embodiments of the inventive concept, the processor may execute the converted machine command on the user terminal.

In some embodiments of the inventive concept, the learned first machine learning model extracts the at least one second biometric signal feature by using an advanced variational autoencoder.

In some embodiments of the inventive concept, the learned first machine learning model is composed of an artificial neural network having a form of an advanced variational autoencoder, and is learned such that the at least one first biometric signal feature is input to the artificial neural network and the at least one first biometric signal feature is output as a final result, wherein the artificial neural network is composed of a first recursive neural network acting as an encoder and a second recursive neural network acting as a decoder, wherein an input of the first recursive neural network is the at least one first biometric signal feature, and an output of the first recursive neural network is the at least one second biometric signal feature, and wherein an input of the second recursive neural network is the at least one second biometric signal feature, and an output of the second recursive neural network is the at least one first biometric signal feature.

In some embodiments of the inventive concept, each of a connection between the at least one first biometric signal feature and a unit of the first recursive neural network, and a connection between the at least one second biometric signal feature and a unit of the second recursive neural network is an ALL-TO-ALL linear connection, wherein a connection weight is randomly determined by a uniform distribution, and wherein a value of the connection weight is fixed during an initialization procedure and is not changed afterward.

In some embodiments of the inventive concept, each of a connection between the unit of the first recursive neural network and the at least one second biometric signal feature and a connection between the unit of the second recursive neural network and the at least one first biometric signal feature is an ALL-TO-ALL linear connection, and wherein a value of a connection weight of the connection between the unit of the first recursive neural network and the at least one second biometric signal feature and the connection between the unit of the second recursive neural network and the at least one first biometric signal feature is changed while being learned by a linear learning algorithm.

In some embodiments of the inventive concept, the value of the connection weight of the connection between the unit of the first recursive neural network and the at least one second biometric signal feature and the connection between the unit of the second recursive neural network and the at least one first biometric signal feature is randomly determined by a uniform distribution and then is changed while being learned by the linear learning algorithm.

In some embodiments of the inventive concept, the second machine learning model including a third recursive neural network that uses the at least one second biometric signal as an input and uses the word constituting the thoughts of the user as an output in the learned first machine learning model.

In some embodiments of the inventive concept, a connection between the at least one second biometric signal feature and a unit of the third recursive neural network is an ALL-TO-ALL linear connection, a connection weight is randomly determined by a uniform distribution, and a value of the connection weight is fixed during an initialization procedure and is not changed afterward, wherein a connection between the unit of the third recursive neural network and a unit corresponding to the word constituting the thoughts of the user is an ALL-TO-ALL linear connection, and wherein a value of a connection weight of the connection between the unit of the third recursive neural network and the unit corresponding to the word constituting the thoughts of the user is changed while being learned by a linear learning algorithm.

According to an embodiment, a method for controlling an artificial intelligence system for decoding thoughts of a user may include preprocessing, by a processor of the system, a measured biometric signal, extracting, by the processor, at least one first biometric signal feature from the preprocessed biometric signal, determining, by the processor, at least one second biometric signal feature necessary to identify thoughts of the user among the at least one first biometric signal feature by using a first machine learning model learned to identify thoughts of the user, learning, by the processor, a second machine learning model while using the at least one second biometric signal as an input and using a word constituting the thoughts of the user as an output in the learned first machine learning model, and deriving, by the processor, at least one word constituting the thoughts of the user by using the learned first machine learning model and the learned second machine learning model

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic diagram for collecting learning data, according to an embodiment of the inventive concept;

FIG. 2 is a block diagram illustrating a decoding system, according to an embodiment of the inventive concept;

FIG. 3 is a schematic diagram illustrating decoding of a user's thoughts by using a decoding system, according to an embodiment of the inventive concept; and

FIGS. 4A to 4E are schematic diagrams illustrating decoding of a user's thoughts by using a decoding system, according to an embodiment of the inventive concept.

### DETAILED DESCRIPTION

The above and other aspects, features and advantages of the inventive concept will become apparent from embodiments to be described in detail in conjunction with the accompanying drawings. The inventive concept, however, may be embodied in various different forms, and should not be construed as being limited only to the illustrated embodiments. Rather, these embodiments are provided as examples so that the inventive concept will be thorough and complete, and will fully convey the scope of the inventive concept to those skilled in the art. The inventive concept may be defined by the scope of the claims.

The terms used herein are provided to describe embodiments, not intended to limit the inventive concept. In the specification, the singular forms include plural forms unless particularly mentioned. The terms “comprises” and/or “comprising” used herein do not exclude the presence or addition of one or more other components, in addition to the aforementioned components. The same reference numerals denote the same components throughout the specification. As used herein, the term “and/or” includes each of the associated components and all combinations of one or more of the associated components. It will be understood that, although the terms “first”, “second”, etc., may be used herein to describe various components, these components should not be limited by these terms. These terms are only used to distinguish one component from another component. Thus, a first component that is discussed below could be termed a second component without departing from the technical idea of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

As illustrated in the figures, spatially relative terms, such as “below”, “beneath”, “lower”, “above”, “upper”, and the like, may be used herein for ease of description to describe the relationship between one component and other components. It will be understood that the spatially relative terms are intended to encompass different orientations of the components in use or operation in addition to the orientation depicted in the figures. For example, when inverting a component shown in the figures, a component described as “below” or “beneath” of another component may be placed “above” another element. Thus, the exemplary term “below” may include both downward and upward directions. The components may also be oriented in different directions, and thus the spatially relative terms may be interpreted depending on orientation.

Hereinafter, an embodiment of the inventive concept will be described in detail with reference to the accompanying drawings.

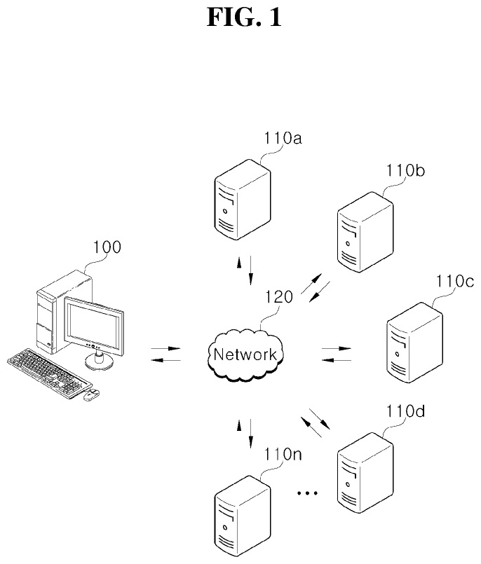

FIG. 1 is a schematic diagram for collecting learning data, according to an embodiment of the inventive concept.

A decoding system 100 according to an embodiment of the inventive concept may provide an artificial intelligence system that decodes a user's thoughts. The decoding system 100 may communicate with at least one server 110a, 110b, 110c, 110d, . . . , or 110n to collect learning data for learning. In this case, the at least one server 110a, 110b, 110c, 110d, . . . , or 110n may include a cloud server such as Amazon Web Services (AWS) or MS Azure. In addition, the at least one server 110a, 110b, 110c, 110d, . . . , or 110n may include a calculation server that identifies words constituting the user's thoughts by analyzing biometric signals. Accordingly, the decoding system 100 may obtain analysis data of various biometric signals and the words constituting the user's thoughts corresponding to specific biometric signals from the at least one server 110a, 110b, 110c, 110d, . . . , or 110n.

The decoding system 100 may communicate with at least one server 110a, 110b, 110c, 110d, . . . , or 110n by using a network 120. The network 120 may include a connection unit (not shown) such as a wired or wireless communication link or an optical fiber cable. In addition, the network 120 may also be implemented as various networks such as Intranet, a local area network (LAN), or a wide area network (WAN).

The decoding system 100 according to an embodiment of the inventive concept may output the words constituting the user's thoughts through the user's biometric signal by using a machine learning model, which is learned for identifying the user's thoughts, such as deep learning. Moreover, the decoding system 100 may convert the derived at least one word into a machine command for controlling a user terminal by using a natural language processing scheme (NLP) and may execute the converted machine command on the user terminal.

Here, the user terminal may be various electronic devices controlled by machine commands. For example, the user terminal may be one of electronic devices such as a computer, a ultra-mobile PC (UMPC), a workstation, a net-book, personal digital assistants (PDAs), a portable computer, a web tablet, a wireless phone, a mobile phones, a smart phone, an e-book, a portable multimedia player (PMP), a portable game console, a navigation device, a black box, or a digital camera.

The deep learning may refer to an artificial neural network-based machine learning method that allows a machine to perform learning by simulating human biological neurons.

As an example of a machine learning model, a deep neural network (DNN) may include a system or network that builds one or more layers in one or more computers and performs determination based on pieces of data.

The DNN may be implemented as a set of layers including a convolutional pooling layer, a locally-connected layer, and a fully-connected layer.

The convolutional pooling layer or locally-connected layer may be configured to extract features of a biometric signal. The fully-connected layer may determine the correlation between the features of the biometric signal.

As another example, the overall structure of DNN according to an embodiment of the inventive concept may be configured in a form in which the locally-connected layer is connected to the convolutional pooling layer and the fully-connected layer is connected to the locally-connected layer. The DNN may include various determination criteria (i.e., parameters) and may add a new determination criterion (i.e., a parameter) by analyzing the input biometric signal.

Furthermore, according to an embodiment of the inventive concept, learning data for machine learning may be generated based on a U-Net-dhSegment model. Here, the U-Net-dhSegment model may refer to a model that creates a U-shaped architecture with skip connections for each level by setting an expansive path to be symmetric with a contracting path based on fully convolutional networks (FCN) of end-to-end.

According to an embodiment of the inventive concept, a machine learning model may perform learning to decode words, which constitute the user's thoughts, by using analysis data of various biometric signals and learning data including at least one of the words constituting the user's thoughts corresponding to a specific biometric signal. The machine learning model may use the analysis data of various biometric signals and the words constituting the user's thoughts corresponding to the specific biometric signal as learning data.

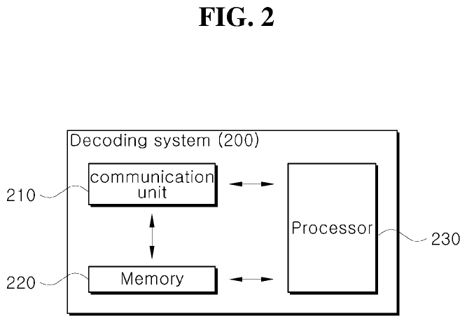

FIG. 2 is a block diagram illustrating a decoding system 200, according to an embodiment of the inventive concept. The decoding system 200 of FIG. 2 may correspond to the decoding system 100 of FIG. 1.

According to an embodiment of the inventive concept, the decoding system 200 may include a communication unit 210, a memory 220, and a processor 230. The components shown in FIG. 2 are not essential in implementing the decoding system 200. The decoding system 200 described herein may have more or fewer components than those listed above. For example, the communication unit 210 among the components may include one or more modules, each of which enables wireless communication with an external device or an external server.

A biometric signal may include at least one of a brain wave signal and a gaze signal, but is not limited thereto. For example, the user's gaze signal may be collected through an eye-tracker.

The brain wave refers to the result recorded by attaching electrodes to a scalp to induce microscopic electrical activities of brain cells and amplifying the induced result by a brain wave system by using an electric potential being a vertical axis and a time being a horizontal axis. In other words, the brain wave may be obtained by measuring electrical activities occurring in a cerebral cortex. The brain wave may change spatio-temporally depending on brain activities, a measurement state, and a brain function and may mainly have a frequency of 0 to 50 Hz and amplitude of 10 to 200 uV. Moreover, the brain wave is classified into a delta wave (δ wave), a theta wave (θ wave), an alpha wave (α wave), and a beta wave (β wave) depending on a frequency range. There may be features of each brain wave for each frequency.

According to an embodiment of the inventive concept, electroencephalogram (EEG) refers to an electrical recording signal recorded by inducing potential fluctuations occurring in a brain of a human or animal on a scalp, or brain currents caused by the potential fluctuations Magnetoencephalogram (MEG) refers to a signal recorded by measuring microscopic biomagnetism generated from electrical activities of brain biological neurons with a SQUID sensor. Electrocorticogram (ECoG) refers to an electrical recording signal recorded by directly measuring potential fluctuations occurring in the cerebrum or brain current caused by the potential fluctuations by implanting electrodes from the surface of the cerebral cortex. According to an embodiment of the inventive concept, the near-infrared spectroscopy (NIRS) brain wave signal refers to a signal obtained by measuring and recording a difference between a low-level light wave and a wave obtained by reflecting the low-level light wave on a brain. In the present specification, the brain wave signals such as EEG, MEG, and ECoG are described by way of example. The brain wave signal is not limited to the specific type of a brain wave signal. For example, the brain wave signal may refer to all signals that are generated from the human brain and are capable of being measured in a human head.

The measured biometric signal may be received through the communication unit 210 of the decoding system 200. According to an embodiment of the inventive concept, the communication unit 210 may communicate with various types of external devices depending on various types of communication methods. The communication unit 210 may include at least one of a Wi-Fi chip, a Bluetooth chip, a wireless communication chip, and an NFC chip.

According to the mobile communication technology of the present specification, a wireless signal is transmitted and received with at least one of a base station, an external terminal, and an external server on a mobile communication network established depending on technical standards or communication methods (e.g., global system for mobile communication (GSM), code division multi access (CDMA), code division multi access 2000 (CDMA2000), enhanced voice-data optimized or enhanced voice-data only (EV-DO), wideband CDMA (WCDMA), high speed downlink packet access (HSDPA), high speed uplink packet access (HSUPA), long term evolution (LTE), long term evolution-advanced (LTE-A), and the like).

Moreover, according to an embodiment of the inventive concept, a short-distance communication technology may include a technology that supports short-distance communication by using at least one of Bluetooth™, radio frequency identification (RFID), infrared data association (IrDA), ultra-wideband (UWB), ZigBee, near field communication (NFC), Wi-Fi, Wi-Fi Direct, and wireless universal serial bus (Wireless USB) technologies.

According to an embodiment of the inventive concept, the memory 220 is a local storage medium supporting various functions of the decoding system 200. In addition to a biometric signal received by the communication unit 210, the memory 220 may store analysis data of a biometric signal, words constituting a user's thoughts corresponding to a specific biometric signal, and the like. Furthermore, the memory 220 may store a plurality of application programs (or applications) running in the decoding system 200, data for an operation of the decoding system 200, and instructions. At least part of the application programs may be downloaded from an external server through wireless communication. The application program may be stored in the memory 220, may be installed in the decoding system 200, and may be driven by the processor 230 to perform an operation (or function) of the decoding system 200.

Moreover, even when the power supply to the decoding system 200 is cut off, data needs to be stored. Accordingly, the memory 220 according to an embodiment of the inventive concept may be provided as a writable non-volatile memory (writable ROM) to reflect changes. That is, the memory 220 may be provided as one of a flash memory, an EPROM, or an EEPROM. For convenience of description in an embodiment of the inventive concept, it is described that all instruction information is stored in the single memory 220. However, an embodiment is not limited thereto. For example, the decoding system 200 may include a plurality of memories.

In addition to an operation associated with the application program, the processor 230 generally controls overall operations of the decoding system 200. The processor 230 may provide or process appropriate information or functions to a user, by processing a signal, data, information, or the like, which is input or output through the above-described components, or driving the application program stored in the memory 220.

Besides, the processor 230 may control at least part of the components described with reference to FIG. 2 to operate the application program stored in the memory 220. Furthermore, the processor 230 may combine and operate at least two or more of the components included in the decoding system 200 to operate the application program.

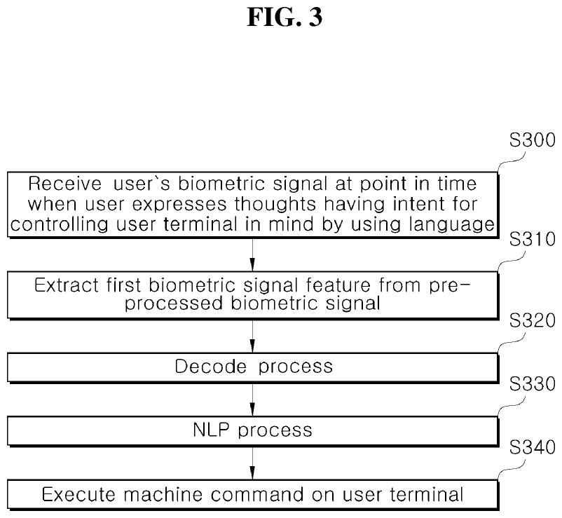

Hereinafter, an operation of the processor 230 of the decoding system 200 will be described with reference to FIGS. 3 to 6.

According to an embodiment of the inventive concept, when a biometric signal is received through the communication unit 210, the processor 230 may perform preprocessing.

In general, when the biometric signal is measured, there may be a section in which noise occurs due to heartbeat or body movements. Accordingly, the processor 230 may perform a preprocessing process of removing unnecessary high-frequency and low-frequency components for diagnosing mental disorders through brain wave analysis and removing artifacts due to movements.

According to an embodiment of the inventive concept, the processor 230 may preprocess the received biometric signal through noise removal or filtering. In particular, to remove noise, the processor 230 may use an independent component analysis or principal component analysis for removing the noise of electromyography (EMG) or electrooculogram (EOG).

In addition, the processor 230 may remove the noise by using one of a low-pass filter, a high-pass filter, a band-pass filter, and a notch filter. For example, in addition to the general noise signal according to a general transmission path (wired and wireless channels), other biometric signals other than brain wave signals such as EMG and EOG are not interest signals, the processor 230 may regard the signals as noise and may remove the noise through filtering.

Also, according to an embodiment of the inventive concept, an epoching process refers to cutting noise-free brain wave data with a specific section so as to be processed. The epoching may be used during an interval between tens of milliseconds and seconds.

According to an embodiment of the inventive concept, the processor 230 may extract at least one biometric signal feature from the preprocessed biometric signal. In this case, the processor 230 may extract power for each frequency through spectral density analysis, and may extract quantitative biometric signal features by using linear, nonlinear, or complex network analysis.

The brain wave signal will be described as a biometric signal. According to an embodiment of the inventive concept, a first brain wave extracted through the processor 230 may include the power, frequency, and inter-channel connectivity of a brain wave (e.g., γ wave, α wave, β wave, δ wave, θ wave, or the like) for each frequency of the user. Here, the channel may include a plurality of points on a scalp where the user's brain wave is measured. In addition, the inter-channel connectivity may include a phase locking value (PLV), correlation coefficient, coherence coefficient, Granger's causality index, partial directed coherence (PDC), directed transfer function (DTF), mutual information, transfer entropy, and synchronization likelihood between brain wave signals.

When at least one first biometric signal feature is extracted, the processor 230 may determine at least one second biometric signal feature required to identify the user's intent, by using a first machine learning model learned for identifying the user's thoughts.

Besides, the processor 230 may learn a second machine learning model by using the at least one second biometric signal as an input and by using words composing the user's thoughts as an output in the first machine learning model thus learned, and may derive at least one word constituting the user's thoughts by using the first and second machine learning models thus learned.

Besides, the processor 230 may convert the derived at least one word into a machine command for controlling a user terminal by using a natural language processing scheme (NLP). Moreover, the processor 230 may execute the converted machine command on the user terminal.

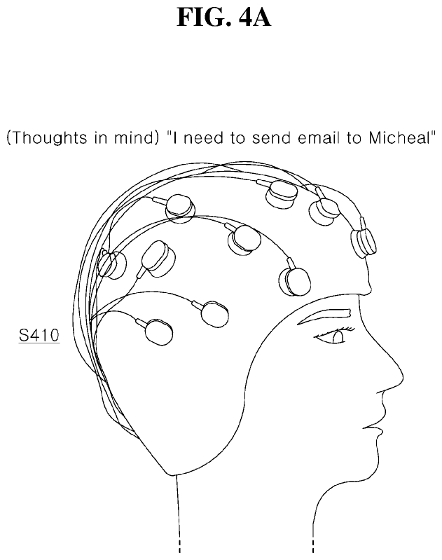

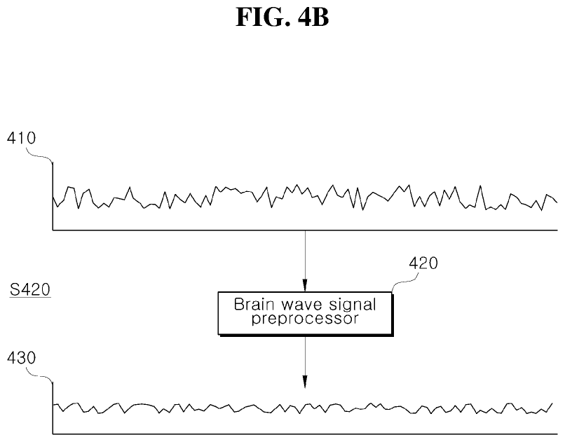

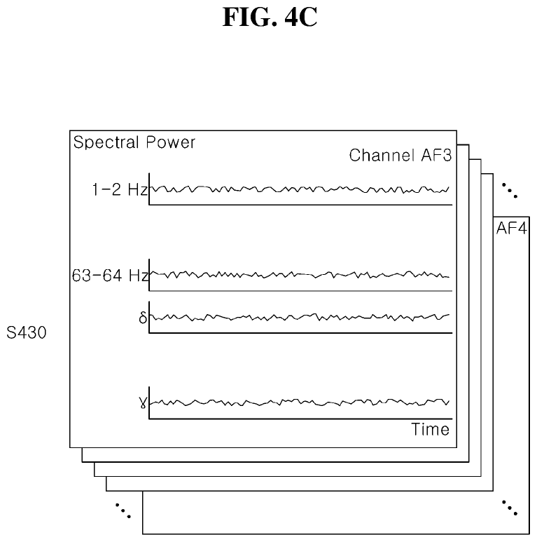

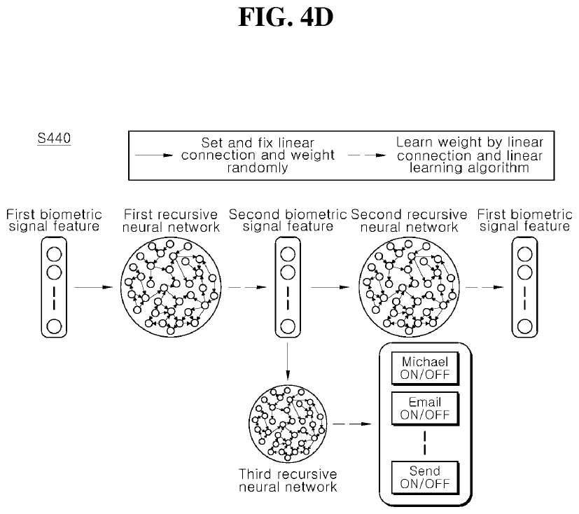

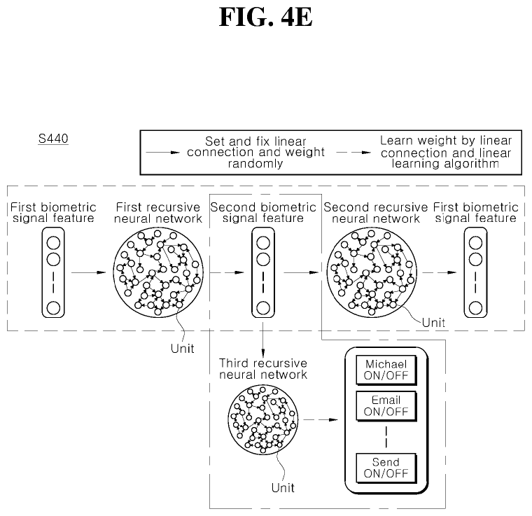

FIG. 3 is a schematic diagram illustrating decoding of a user's thoughts by using a decoding system, according to an embodiment of the inventive concept. For convenience of description, FIGS. 4A to 4E will be referred to together.

First of all, when a user expresses thoughts having intent for controlling a user terminal in mind by using a language. The user's biometric signal is captured and collected when the user expresses thoughts having intent for controlling the user terminal in mind by using a language. Here, the biometric signal may include at least one of a brain wave signal and a gaze signal.

The decoding system 200 according to an embodiment of the inventive concept receives the user's biometric signal at a point in time when the user expresses thoughts having intent for controlling the user terminal in mind by using a language [S300]. For example, referring to FIG. 4A, the user may express thoughts having intent for controlling the user terminal in mind and saying that “I need to send an email to Michael”. At this time, the user's biometric signal may be collected.

Here, a method of the decoding system 200 receiving the user's biometric signal is not limited thereto.

Next, the processor 230 may extract the first biometric signal feature by performing preprocessing and feature extraction processes on the user's biometric signal [S310].

Here, the first biometric signal feature means a quantitative feature of the user's biometric signal thus preprocessed. For example, when the user's biometric signal includes a brain wave signal, a first brain wave feature may include the power, frequency, and inter-channel connectivity of a brain wave (e.g., γ wave, α wave, β wave, δ wave, θ wave, or the like) for each frequency.

Next, the decoding system 200 may output words constituting the user's thoughts through the decoding process by using the first biometric signal feature as an input [S320]. Here, the decoding process may refer to a series of processing procedures for outputting words constituting the user's thoughts by using a machine learning model of the present specification.

The decoding system 200 may output key words constituting the user's thoughts through the decoding process. For example, there may be words indicating a subject, a verb, an object, or the like as key words constituting the user's thoughts. For example, through the decoding process, the decoding system 200 may output words “Michael”, “email”, and “send” as key words constituting the user's thoughts.

When the key words constituting the user's thoughts are completely output, the decoding system 200 may convert the derived at least one word into a machine command for controlling a user terminal by using a natural language processing scheme (NLP) [S330].

Next, the decoding system 200 may allow the converted machine command to be executed on the user terminal [S340].

The decoding system 200 may directly execute the converted machine command in the user terminal or may transmit the machine command to the user terminal such that the user terminal executes the converted machine command.

In this way, the decoding system 200 may not only decode the thoughts having the user's intent, but may also control the user terminal depending on the decoded thoughts having the user's intent, based on a brain machine interface (BMI) technology.

Hereinafter, a process of decoding the user's thoughts will be described in more detail with reference to FIGS. 4A to 4E.

FIGS. 4A to 4E are schematic diagrams illustrating decoding of a user's thoughts by using a decoding system, according to an embodiment of the inventive concept.

Above all, referring to FIG. 4A, a user's brain wave may be measured as a biometric signal through a brain wave measuring device [S410].

A patient's brain wave may be measured by the brain wave measuring device such as Emotiv, OpenBci, NeuroSky or Geodesic™ and then may be transmitted to the communication unit 210 of the decoding system 200 through a network.

Referring to FIG. 4B, a brain wave 410 thus received may be preprocessed through noise removal and epoching processing [S420].

In detail, the brain wave 410 thus received may be preprocessed through noise removal and epoching processing by a brain wave signal preprocessor 420 that removes noise by using at least one of a low-pass filter, a high-pass filter, a band-pass filter, and a notch filter. In this case, when the decoding system 200 includes the brain wave signal preprocessor 420, the processor 230 may perform preprocessing by controlling the brain wave signal preprocessor 420. Alternatively, when the brain wave signal preprocessor 420 is an external device of the decoding system 200, the decoding system 200 may receive a brain wave 430, which is preprocessed by the brain wave signal preprocessor 420, through the communication unit 210.

Referring to FIG. 4C, when the preprocessing is completed, the processor 230 may extract at least one first biometric signal feature from the preprocessed brain wave 430 [S430].

In detail, the processor 230 may extract power for each frequency through spectral density analysis, and may extract quantitative brain wave features by using linear, nonlinear, or complex network analysis.

Next, referring to FIGS. 4D and 4E, the processor 230 may input the extracted at least one first biometric signal feature to the machine learning model learned for identifying the user's thoughts and may output words constituting the user's thoughts [S440]. S440 describes the decoding process of S320 in detail.

According to an embodiment of the inventive concept, the processor 230 may determine at least one second biometric signal feature required to identify the user's intent from among the at least one first biometric signal feature.

Here, the second biometric signal feature may include the power, frequency, and inter-channel connectivity of a brain wave (e.g., γ wave, α wave, β wave, δ wave, θ wave, or the like) for each frequency of a patient.

To determine the at least one second biometric signal feature required to identify the user's intent from among the at least one first biometric signal feature, the processor 230 may use a machine learning model learned through an advanced variational autoencoder, which will be described later.

Here, the machine learning model according to an embodiment of the inventive concept will be described in detail with reference to FIGS. 4D and 4E.

The machine learning model according to an embodiment of the inventive concept includes the first and second machine learning models. According to an embodiment of the inventive concept, the processor 230 may determine at least one second biometric signal feature required to identify the user's intent from among the at least one first biometric signal feature by using the first machine learning model. Moreover, the second machine learning model may perform learning while using the at least one second biometric signal of the first machine learning model as an input and using the words constituting the user's thoughts as an output. Here, the first and second machine learning models may be learned at the same time or may be learned step by step.

The first machine learning model according to an embodiment of the inventive concept is composed of an artificial neural network having a form of an advanced variational autoencoder, and is learned such that at least one first biometric signal feature extracted from the biometric signal including the preprocessed brain wave 430 is input to the artificial neural network and is output as a final result at the same time.

In particular, the artificial neural network of the first machine learning model is composed of a first recursive neural network acting as an encoder and a second recursive neural network acting as a decoder. The input of a first recursive neural network is at least one first biometric signal feature, and the output of the first recursive neural network is at least one second biometric signal feature. The input of a second recursive neural network is at least one second biometric signal feature, and the output of the second recursive neural network is at least one first biometric signal feature.

That is, in an artificial neural network of the first machine learning model, at least one first biometric signal feature is given as an input to the first recursive neural network, and at least one first biometric signal feature is learned to be the output of the artificial neural network of the first machine learning model. In this learning process, at least one second biometric signal feature is automatically learned on the way. Meanwhile, at least one second biometric signal feature is used as an input to a third recursive neural network, which will be described later.

The first recursive neural network is composed of a plurality of units. The number (N) of units constituting the first recursive neural network is determined by the number (M) of first biometric signal features. For example, the number (N) of units constituting the first recursive neural network may be determined by the processor 230 to be greater than 100 times the number (M) of first brain wave features (N>M*100).

Here, when the number of units (N) constituting the first recursive neural network increases, the performance of the artificial neural network increases. However, because the learning process takes a long time, the number (N) of units constituting the first recursive neural network in this artificial neural network is set to be greater than 100 times the number (M) of first biometric signal features.

In addition, the units constituting the first recursive neural network are randomly and recursively connected to each other. In this case, for example, the processor 230 may set a connection probability between units to a value between 0.1% and 1%.

Besides, for example, a connection weight between units constituting the first recursive neural network is determined by a uniform distribution among values between −1 and 1. The processor 230 may multiply connection weight values by a specific scaling factor such that an absolute value of the greatest eigen value of a connection weight matrix (W) thus determined afterward is not greater than, for example, 1.

The connection weight matrix (W) calculated in such the manner is fixed and is not changed.

The second recursive neural network is also formed under the same conditions as the first recursive neural network. Also, the connection weight matrix (W) calculated for the second recursive neural network is fixed and is not changed.

In the meantime, each of a connection between the first biometric signal feature and the unit of the first recursive neural network, and a connection between the second biometric signal feature and the unit of the second recursive neural network may be an ALL-TO-ALL linear connection. The connection weight may be randomly determined by a uniform distribution among values between −1 and 1, for example. A value of the connection weight is fixed during an initialization procedure and is not changed afterward.

Besides, each of a connection between the unit of the first recursive neural network and the second biometric signal feature and a connection between the unit of the second recursive neural network and the first biometric signal feature is also an ALL-TO-ALL linear connection. In an initialization process, a connection weight between the connection between the unit of the first recursive neural network and the second biometric signal feature, and the connection between the unit of the second recursive neural network and the first biometric signal feature may be randomly determined by a uniform distribution among values between −1 and 1, for example.

However, a value of the connection weight between the connection between the unit of the first recursive neural network and the second biometric signal feature, and the connection between the unit of the second recursive neural network and the first biometric signal feature is not fixed to a value determined during the initialization process, but changed while being learned by a linear learning algorithm. For example, a linear regression or pseudo inverse matrix method may be used as the linear learning algorithm.

Because the connection relationship is not a linear connection when the first machine learning model is composed of an artificial neural network having a form of an autoencoder or variational autoencoder, not an advanced variational autoencoder, the computational complexity is increased. As a result, a running rule becomes complicated. Besides, in the case of an artificial neural network having a form of a variational autoencoder or autoencoder, there are also machine restrictions that require the use of high-performance machines depending on the complexity of the learning rule.

On the other hand, a machine learning model according to an embodiment of the inventive concept may set and fix weights of some configurations randomly based on a linear connection and may learn only weights of the second biometric signal feature and the first biometric signal feature based on a linear learning algorithm, thereby obtaining high accuracy based on a reservoir computing method while the computational complexity is reduced.

In addition, because the decoding system 200 according to an embodiment of the inventive concept uses a linear connection and a linear learning algorithm, the decoding system 200 according to an embodiment of the inventive concept may reduce the learning amount and may calculate an explainable result. In detail, because the learned result from the first recursive neural network to the second biometric signal feature, the second recursive neural network, and the biometric signal feature in FIG. 4D are linearly connected in the decoding system 200 according to an embodiment of the inventive concept, the learned results from the first recursive neural network to the second biometric signal feature, the second recursive neural network, and the first biometric signal feature through the filter may be expressed as a first-order equation.

The second machine learning model may use the at least one second biometric signal as an input and may use words constituting the user's thoughts as an output. The artificial neural network of the second machine learning model includes a third recursive neural network. In the learned first machine learning model, the third recursive neural network uses at least one second biometric signal as an input and uses the words composing the user's thoughts as an output.

In detail, the third recursive neural network is also composed under the same conditions as the first recursive neural network and the second recursive neural network. The connection weight matrix (W) calculated for the third recursive neural network is fixed and is not changed.

In the meantime, a connection between the second biometric signal feature and the unit of the third recursive neural network may be an ALL-TO-ALL linear connection. The connection weight may be randomly determined by a uniform distribution among values between −1 and 1, for example. A value of the connection weight is fixed during an initialization procedure and is not changed afterward.

Besides, a connection between the unit of the third recursive neural network and a unit corresponding to a word constituting the user's thoughts is also an ALL-TO-ALL linear connection. In an initialization process, a connection weight of the connection between the unit of the third recursive neural network and the unit corresponding to the word constituting the user's thoughts may be randomly determined by a uniform distribution among values between −1 and 1, for example.

However, the connection weight between the unit of the third recursive neural network and the unit corresponding to the word constituting the user's thoughts is not fixed to a value determined during the initialization process, but changed while being learned by a linear learning algorithm. For example, a linear regression or pseudo inverse matrix method may be used as the linear learning algorithm.

The third recursive neural network is learned to distinguish words to be decoded in this system. For example, when a word of “email” is present in words thought by the user, a second unit among outputs of the third recursive neural network of FIG. 4D is turned on through this process. When there are several words thought by the user, some units corresponding to words thought by the user are turned on and the other units are turned off.

Accordingly, in the machine learning model according to an embodiment of the inventive concept, the NLP process (S330) is performed on a word corresponding to a unit thus turned on. The processor 230 may perform natural language processing on the derived at least one word by using a natural language processing scheme (NLP) and may convert the natural language processing result into a machine command for controlling the user terminal.

The natural language processing scheme (NLP) may refer to the overall technology that mechanically analyzes language phenomena and makes the analyzed result into a form understood by the processor 230, and, for example, may refer to a scheme capable of syntactic analysis to determine the structure of a phrase or clause by identifying components of a clause and analyzing the hierarchical relationship of these components.

The processor 230 may perform natural language processing on the derived main words (a subject, a verb, an object, and the like) by using a natural language processing scheme (NLP) and may identify the user's thoughts having the intent for controlling the user terminal through the processed result.

Moreover, the processor 230 may convert the processed result into a machine command for controlling the user terminal. To derive the machine command for controlling the user terminal depending on the identified thoughts of the user, the corresponding machine command may be pre-stored in the memory 220 or an external server.

Moreover, the processor 230 may cause the converted machine command to be executed on the user terminal.

The decoding system 200 may directly execute the converted machine command in the user terminal or may transmit the machine command to the user terminal such that the user terminal executes the converted machine command.

For example, the processor 230 may allow the user terminal to send an email to Michael by using the convert machine command.

According to an embodiment of the inventive concept, it is possible to control a user terminal depending on a user's thoughts by identifying the user's thoughts through an artificial intelligence system that decodes the user's thoughts.

Steps or operations of the method or algorithm described with regard to an embodiment of the inventive concept may be implemented directly in hardware, may be implemented with a software module executable by hardware, or may be implemented by a combination thereof. The software module may reside in a random-access memory (RAM), a read only memory (ROM), an erasable programmable ROM (EPROM), an electrically erasable programmable ROM (EEPROM), a flash memory, a hard disk, a removable disk, a CD-ROM, or a computer-readable recording medium well known in the art to which the inventive concept pertains.

Although an embodiment of the inventive concept are described with reference to the accompanying drawings, it will be understood by those skilled in the art to which the inventive concept pertains that the inventive concept may be carried out in other detailed forms without changing the scope and spirit or the essential features of the inventive concept. Therefore, the embodiments described above are provided by way of example in all aspects, and should be construed not to be restrictive.

What is claimed is:

1. An artificial intelligence system for decoding thoughts of a user, the system comprising a processor configured to:
preprocess a measured biometric signal;
extract at least one first biometric signal feature from the preprocessed biometric signal;
determine at least one second biometric signal feature necessary to identify thoughts of the user among the at least one first biometric signal feature by using a first machine learning model learned to identify thoughts of the user;
learn a second machine learning model while using the at least one second biometric signal as an input and using a word constituting the thoughts of the user as an output in the learned first machine learning model; and
derive at least one word constituting the thoughts of the user by using the learned first machine learning model and the learned second machine learning model.

2. The system of claim 1, wherein the processor is configured to:
convert the derived at least one word into a machine command for controlling a user terminal by using a natural language processing scheme (NLP).

3. The system of claim 2, wherein the processor is configured to:
execute the converted machine command on the user terminal.

4. The system of claim 1, wherein the learned first machine learning model extracts the at least one second biometric signal feature by using an advanced variational autoencoder.

5. The system of claim 1, wherein the learned first machine learning model is composed of an artificial neural network having a form of an advanced variational autoencoder, and is learned such that the at least one first biometric signal feature is input to the artificial neural network and the at least one first biometric signal feature is output as a final result,
wherein the artificial neural network is composed of a first recursive neural network acting as an encoder and a second recursive neural network acting as a decoder,
wherein an input of the first recursive neural network is the at least one first biometric signal feature, and an output of the first recursive neural network is the at least one second biometric signal feature, and
wherein an input of the second recursive neural network is the at least one second biometric signal feature, and an output of the second recursive neural network is the at least one first biometric signal feature.

6. The system of claim 5, wherein each of a connection between the at least one first biometric signal feature and a unit of the first recursive neural network, and a connection between the at least one second biometric signal feature and a unit of the second recursive neural network is an ALL-TO-ALL linear connection,
wherein a connection weight is randomly determined by a uniform distribution, and
wherein a value of the connection weight is fixed during an initialization procedure and is not changed afterward.

7. The system of claim 6, wherein each of a connection between the unit of the first recursive neural network and the at least one second biometric signal feature and a connection between the unit of the second recursive neural network and the at least one first biometric signal feature is an ALL-TO-ALL linear connection, and
wherein a value of a connection weight of the connection between the unit of the first recursive neural network and the at least one second biometric signal feature and the connection between the unit of the second recursive neural network and the at least one first biometric signal feature is changed while being learned by a linear learning algorithm.

8. The system of claim 7, wherein the value of the connection weight of the connection between the unit of the first recursive neural network and the at least one second biometric signal feature and the connection between the unit of the second recursive neural network and the at least one first biometric signal feature is randomly determined by a uniform distribution and then is changed while being learned by the linear learning algorithm.

9. The system of claim 1, wherein the second machine learning model including a third recursive neural network that uses the at least one second biometric signal as an input and uses the word constituting the thoughts of the user as an output in the learned first machine learning model.

10. The system of claim 9, wherein a connection between the at least one second biometric signal feature and a unit of the third recursive neural network is an ALL-TO-ALL linear connection, a connection weight is randomly determined by a uniform distribution, and a value of the connection weight is fixed during an initialization procedure and is not changed afterward,
wherein a connection between the unit of the third recursive neural network and a unit corresponding to the word constituting the thoughts of the user is an ALL-TO-ALL linear connection, and
wherein a value of a connection weight of the connection between the unit of the third recursive neural network and the unit corresponding to the word constituting the thoughts of the user is changed while being learned by a linear learning algorithm.

11. A method for controlling an artificial intelligence system for decoding thoughts of a user, the method comprising:
preprocessing, by a processor of the system, a measured biometric signal;
extracting, by the processor, at least one first biometric signal feature from the preprocessed biometric signal;
determining, by the processor, at least one second biometric signal feature necessary to identify thoughts of the user among the at least one first biometric signal feature by using a first machine learning model learned to identify thoughts of the user;
learning, by the processor, a second machine learning model while using the at least one second biometric signal as an input and using a word constituting the thoughts of the user as an output in the learned first machine learning model; and
deriving, by the processor, at least one word constituting the thoughts of the user by using the learned first machine learning model and the learned second machine learning model.

12. The method of claim 11, further comprising:
converting, by the processor, the derived at least one word into a machine command for controlling a user terminal by using a natural language processing scheme (NLP).

13. The method of claim 12, further comprising:
executing, by the processor, the converted machine command on the user terminal.

14. The method of claim 11, wherein the learned first machine learning model extracts the at least one second biometric signal feature by using an advanced variational autoencoder.

15. The method of claim 11, wherein the learned first machine learning model is composed of an artificial neural network having a form of an advanced variational autoencoder, and is learned such that the at least one first biometric signal feature is input to the artificial neural network and the at least one first biometric signal feature is output as a final result,
wherein the artificial neural network is composed of a first recursive neural network acting as an encoder and a second recursive neural network acting as a decoder,
wherein an input of the first recursive neural network is the at least one first biometric signal feature, and an output of the first recursive neural network is the at least one second biometric signal feature, and
wherein an input of the second recursive neural network is the at least one second biometric signal feature, and an output of the second recursive neural network is the at least one first biometric signal feature.

16. The method of claim 15, wherein each of a connection between the at least one first biometric signal feature and a unit of the first recursive neural network, and a connection between the at least one second biometric signal feature and a unit of the second recursive neural network is an ALL-TO-ALL linear connection,
wherein a connection weight is randomly determined by a uniform distribution, and
wherein a value of the connection weight is fixed during an initialization procedure and is not changed afterward.

17. The method of claim 16, wherein each of a connection between the unit of the first recursive neural network and the at least one second biometric signal feature and a connection between the unit of the second recursive neural network and the at least one first biometric signal feature is an ALL-TO-ALL linear connection, and
wherein a value of a connection weight of the connection between the unit of the first recursive neural network and the at least one second biometric signal feature and the connection between the unit of the second recursive neural network and the at least one first biometric signal feature is changed while being learned by a linear learning algorithm.

18. The method of claim 17, wherein the value of the connection weight of the connection between the unit of the first recursive neural network and the at least one second biometric signal feature and the connection between the unit of the second recursive neural network and the at least one first biometric signal feature is randomly determined by a uniform distribution and then is changed while being learned by the linear learning algorithm.

19. The method of claim 11, wherein the second machine learning model including a third recursive neural network that uses the at least one second biometric signal as an input and uses the word constituting the thoughts of the user as an output in the learned first machine learning model.

20. The method of claim 19, wherein a connection between the at least one second biometric signal feature and a unit of the third recursive neural network is an ALL-TO-ALL linear connection, a connection weight is randomly determined by a uniform distribution, and a value of the connection weight is fixed during an initialization procedure and is not changed afterward,
wherein a connection between the unit of the third recursive neural network and a unit corresponding to the word constituting the thoughts of the user is an ALL-TO-ALL linear connection, and
wherein a value of a connection weight of the connection between the unit of the third recursive neural network and the unit corresponding to the word constituting the thoughts of the user is changed while being learned by a linear learning algorithm.
